(19) United States
(12) Patent Application Publication
(10) Pub. No.: US 2017/0113032 A1
(43) Pub. Date: Apr. 27, 2017

(54) Feedthrough of a Medical Electronic Device, and Medical Electronic Device

(71) Applicant: BIOTRONIK SE & Co. KG, Berlin (DE)

(72) Inventor: Thomas Sontheimer, Rosstal (DE)

(21) Appl. No.: 15/275,733

(22) Filed: Sep. 26, 2016

(30) Foreign Application Priority Data
Oct. 21, 2015 (DE) 10 2015 117 935.0

(51) Int. Cl. A61N 1/02; A61N 1/05; H02G 3/22

(52) U.S. Cl. CPC A61N 1/02; H02G 3/22; A61N 1/0541

(57) ABSTRACT

A feedthrough of a medical electronic device, in particular an implantable medical electronic device, comprising a housing and at least one electric or electronic component received in the housing, wherein the feedthrough has a feedthrough flange for closing an opening of the housing and for supporting at least one connection element, which serves for the connection of the or at least one component externally of the housing, in an insulating element surrounding the connection element, and a grounding pin in mechanical and electrical contact with the feedthrough flange is provided in order to realize a ground connection of the device, wherein the grounding pin is fixed exclusively by form fit and force fit in the feedthrough flange or the insulating element, whilst contacting the feedthrough flange.

### CROSS REFERENCE TO RELATED APPLICATIONS

This patent application claims the benefit of and priority to co pending German Patent

Application No. DE 10 2015 117 935.0, filed on Oct. 21, 2015 in the German Patent Office, which is hereby incorporated by reference in its entirety.

### TECHNICAL FIELD

The present invention relates to a feedthrough of a medical electronic device, in particular an implantable medical electronic device, having a housing and at least one electric or electronic component received in the housing, wherein the feedthrough has a feedthrough flange for closing an opening of the housing and for supporting at least one connection element, which serves for the connection of the or at least one component externally of the housing, in an insulating element surrounding the connection element, and a grounding pin in mechanical and electrical contact with the feedthrough flange is provided in order to realize a ground connection of the device. The present invention also relates to a method for producing a feedthrough of this type and also to a medical electronic device which has a feedthrough of this type.

### BACKGROUND

Implantable devices of the above-mentioned type have long been used on a mass scale, in particular as cardiac pacemakers or implantable cardioverters (in particular, defibrillators).

However, they may also be less complex devices, such as, for example, an electrode or sensor lead. Besides the use of feedthroughs in devices for heart therapy, feedthroughs are also used in cochlear implants.

The implantable electromedical devices of most practical significance are intended to deliver electrical pulses to excitable body tissue via suitably placed electrodes. In order to perform this function, electronic/electrical function units for generating the pulses and for suitably controlling the pulse generation are accommodated in the housing of the device, and electrodes or connections for at least one electrode lead, in the distal end portion of which the electrodes for pulse transfer to the tissue are fitted, are provided directly externally on the device. The electronic/electrical function units in the device interior are to be connected to the external electrodes or electrode lead connections in a way that ensures fail-safe and permanently reliable function under the specific conditions of the implanted state. The connections or electrode lines can also be used to purposefully measure electrical pulses and stimuli in the body of the patient and to record or evaluate these over a relatively long period of time in order to select an individually tailored therapy and to monitor the success of the treatment.

In particular, feedthroughs of which the main and insulating body consists substantially of ceramic or glass are known, wherein multi-layered or multi-part structures with use of metals or metal oxides have also been developed and used. Known feedthroughs of this type largely meet the requirements placed thereon of hermeticity, biocompatibility, signal transfer, and long-term stability.

In order to provide a ground potential for the electronic/electric components and modules of the device, a connection to the metal housing thereof is produced, more specifically, typically by a specific ground connection means in the region of the feedthrough, particularly what is known as a grounding pin. A grounding pin, for example, made of niobium or Pt/Ir, is joined to the housing made of titanium by means of resistance welding in some types of known devices. The electrical connection between housing and circuit board is established after the welding or soldering of the grounding pin to the circuit board and after the welding of the flange to the housing. Alternatively, the ground connection is established by means of a pin which, as it is being fitted, is assembled in a blind bore and is soldered to the flange in a high-temperature soldering process. The electrical connection through the housing is established after the soft soldering of the feedthrough on the circuit board and after the welding of the flange to the housing.

The actual welding process of the grounding pin is less satisfactory in respect of its process stability. On account of the different melting points of the joining partners Nb and Ti, an attachment of the Nb pin to the Ti flange by means of welding is rather unsuitable. The current production of the ground contact in certain variants of the feedthrough by means of a high-temperature soldering process requires fitting time and additional solder material in the form of, for example, gold. Since the grounding pin must be soldered into the metal flange together with the actual soldering (of pins into the insulating ceramic and of the ceramic into the flange), the process window for the soldering process is heavily limited and an optimization of the soldering profile for the actual soldering of the ceramic is hindered. It can be determined that the grounding pin, on account of its design, is usually the hottest point during the soldering, which leads to secondary effects and in some feedthrough types is reflected in the form of metal evaporation. This leads to unnecessary, subsequent cleaning processes.

The present invention is directed toward overcoming one or more of the above-mentioned problems.

### SUMMARY

An object of the present invention is to provide an improved feedthrough of an implantable electromedical device, with which, in particular, the material-related problems of the known welding and soldering methods are avoided and, as a result, the production process and the feedthrough formed herein are less susceptible to faults. At the same time, the production cost is to be kept minimal. Furthermore, a suitable method for producing a feedthrough of this type as well as an improved implantable medical electronic device will be specified.

At least this object is achieved in a first device aspect by a feedthrough having the features of claim 1, and in accordance with a second device aspect by a device having the features of claim 13, and in its method aspect by a method having the features of claim 9. Expedient developments of the inventive concept are disclosed in the dependent claims.

The present invention includes the concept of a deliberate avoidance of an integrally bonded connection between the grounding pin and the surrounding or spatially associated feedthrough part. The present invention also includes the concept of replacing this integrally bonded connection by a combination of a form-locked and force-locked connection. On the whole, this leads to the teaching that the grounding pin is fixed in the feedthrough flange or the insulating element exclusively by means of a form fit and force fit, whilst contacting the feedthrough flange.

Due to the direct insertion of the grounding pin into the flange during the flange production, additional joining processes in the form of grounding pin welding or additional fitting efforts (in the case of high-temperature soldering) and additional material requirements are spared. Furthermore, based on the specific process management mentioned in the introduction, an optimization of the soldering profile to the joining partners (ceramic and metal) without the hotspot constituted by the grounding pin can take place, which results in an increased process stability of the high-temperature soldering process and can help to prevent causal secondary faults, such as evaporation. This could in turn eradicate the need for post-processing steps, which are costly and in turn lead to other, undesirable secondary effects.

In one embodiment of the present invention, the insulating element is formed as a solid ceramic insulating body, and the grounding pin is sintered into the insulating body. In terms of the method, this embodiment is designed such that the insulating element has a recess matched to the form of the grounding pin, the grounding pin in the “green” state of the likewise “green” insulating body is inserted into said body, and the insulating body with inserted grounding pin is finished in a sintering method, preferably together with the “green” flange.

A further embodiment is characterized in that the feedthrough flange or the insulating element comprises a plastic injection-molded part and the grounding pin is overmolded by the plastic injection-molded part. This embodiment is realized in respect of the method such that the grounding pin is placed in an injection mold in order to form the feedthrough flange or insulating element and is overmolded by a plastic material introduced into the mold.

In yet a further embodiment, provision is made for the feedthrough flange to have a flange body made of a first material and for the grounding pin to be formed from a second material, which has a lower coefficient of thermal expansion than the first material, and for the grounding pin to be shrunk into the flange body. In one embodiment, the flange body is a metal body made of a first metal, in particular titanium, and the grounding pin consists of a second metal, which has a lower coefficient of thermal expansion than the first metal, in particular consists of niobium. This embodiment is realized in terms of the method such that the grounding pin is placed in a metal injection mold in order to form the feedthrough flange and the mold is then filled with a liquefied metal, and the grounding pin is overmolded by the metal.

In another embodiment of the above-mentioned design, the grounding pin is embedded in the flange body by heat-shrinking the flange body onto the grounding pin. The associated production method makes provision for a pre-fabricated feedthrough flange made the first metal, in which a recess for receiving the grounding pin is formed, to be heated and inserted in the heated state of the grounding pin into the recess. The feedthrough flange with inserted grounding pin is then cooled in such a way that the grounding pin is shrunk into the feedthrough flange.

Further embodiments, features, aspects, objects, advantages, and possible applications of the present invention could be learned from the following description, in combination with the Figures, and the appended claims.

### DESCRIPTION OF THE DRAWINGS

Advantages and expedient features of the present invention will also become clear from the following description of an exemplary embodiment provided with reference to the drawings, in which:

FIG. 1 shows a schematic, partially sectional illustration of an implantable electromedical device, and

FIGS. 2A and 2B show schematic diagrams of feedthroughs (plan view) in order to explain an exemplary embodiment of the present invention.

### DETAILED DESCRIPTION

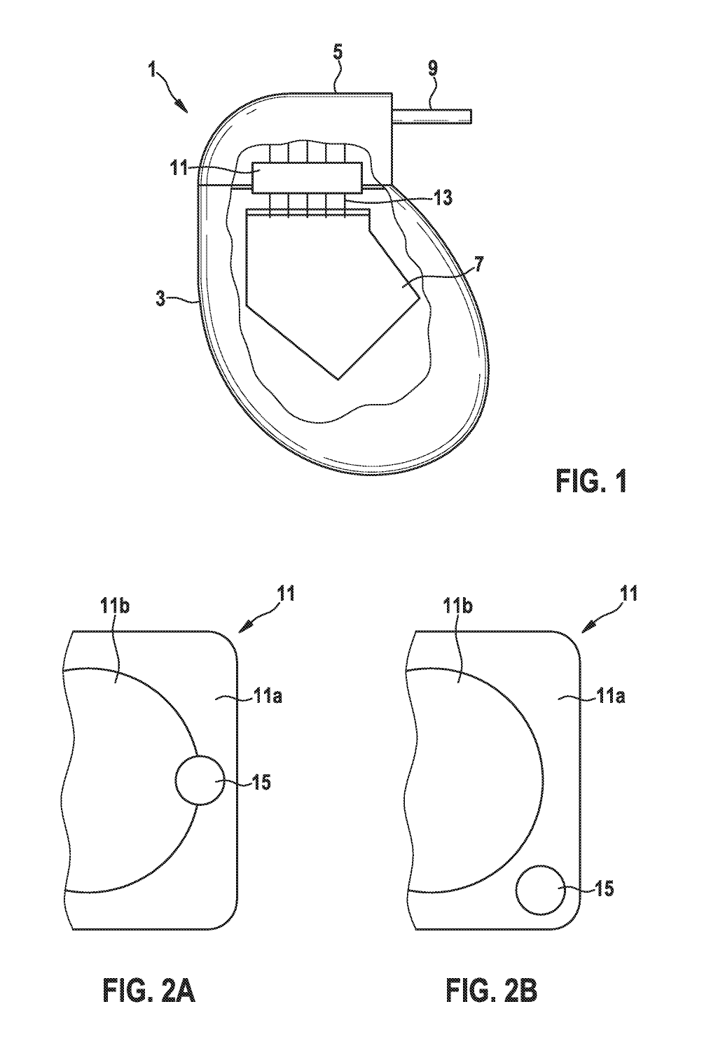

FIG. 1 shows a cardiac pacemaker 1 with a pacemaker housing 3 and a head part (header) 5, in the interior of which a printed circuit board (PCB) 7, in addition to other electronic components, is arranged, an electrode lead 9 being connected to the lead connection (not shown) arranged in the header of said pacemaker. A feedthrough 11 provided between the device housing 3 and the header 5 and comprises a plurality of connection pins 13. The connection pins 13 are fitted at one end through a corresponding bore into the printed circuit board and are soft-soldered thereto.

FIGS. 2A and 2B show schematic sketches (plan views) of two feedthroughs 11 deviating slightly from one another in order to explain exemplary embodiments of the present invention.

In the case of the configuration according to FIG. 2A, the feedthrough 11 comprises a feedthrough flange Ila and a solid insulating body llb arranged within the flange, and an approximately semi-circular recess is formed in both components in such a way that, when the feedthrough flange 11a and insulating body 11b are assembled, a receiving space which is circular in plan view is provided for a grounding pin 15. By contrast, in the embodiment according to FIG. 2B, the receiving space for the grounding pin 15 is formed exclusively in the feedthrough flange 11a, such that the grounding pin is placed at a distance from the insulating body 11b.

The embodiment according to FIG. 2A can have, in principle, a ceramic insulating body 11b or an insulating body manufactured in part from plastic. In the former case, the grounding pin 15 can be inserted prior to the sintering of the insulating body 11b into the semi-circular recess thereof and can be connected in a form-locked and force-locked manner to the insulating body as a result of the sintering process. The grounding pin is then incorporated into the feedthrough flange 11a together with the insulating body as a result of processes known per se.

If, however, the insulating body consists of a plastic material, at least in the portion where the grounding pin 15 is arranged, the grounding pin 15 can be placed at the corresponding point of the injection mold and overmolded by the plastic material so that in this case as well it forms a cohesive structural unit together with the insulating body and this can be incorporated into the feedthrough flange 11a in a subsequent step.

In the configuration according to FIG. 2B, the following process sequences (inter alia) are possible:

In the case of a metal injection molding (MiM) process: The grounding pin is placed in the injection mold of the flange and overmolded by the flange material. The material titanium is preferably used as flange material, and the material niobium is preferably used as pin material. Due to the higher coefficient of thermal expansion of titanium, the flange is shrunk onto the niobium pin during the cooling after the injection molding.

Alternatively, a niobium pin can also be placed in an additively/generatively printed titanium flange before sintering (into the “green” part or “brown” part). As a result of the de-binding/sintering, a form fit is then formed between the pin and the flange.

In a further alternative, the grounding pin can be inserted after the production of a prefabricated (for example, milled) flange 11a: by heating the Ti flange to Ts>T>1200° C. and inserting the Nb pin into the pre-fabricated opening of the hot feedthrough flange, a form fit is provided which also withstands the re-heating by the high-temperature soldering process.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/we claim:

1. A feedthrough of a medical electronic device, in particular an implantable medical electronic device, comprising:
a housing; and
at least one electric or electronic component received in the housing, wherein the feedthrough has a feedthrough flange for closing an opening of the housing and for supporting at least one connection element, which serves for the connection of the or at least one component externally of the housing, in an insulating element surrounding the connection element, and a grounding pin in mechanical and electrical contact with the feedthrough flange is provided in order to realize a ground connection of the device,
wherein the grounding pin is fixed exclusively by form fit and force fit in the feedthrough flange or the insulating element, whilst contacting the feedthrough flange.

2. The feedthrough according to claim 1, wherein the insulating element is formed as a solid ceramic insulating body and the grounding pin is sintered into the insulating body.

3. The feedthrough according to claim 1, wherein the feedthrough flange or the insulating element comprises a plastic injection-molded part and the grounding pin is overmolded by the plastic injection-molded part.

4. The feedthrough according to claim 1, wherein the feedthrough flange has a flange body made of a first material and the grounding pin is formed from a second material, which has a lower coefficient of thermal expansion than the first material, and the grounding pin is shrunk into the flange body.

5. The feedthrough according to claim 4, wherein the flange body is a metal body made of a first metal, in particular titanium, and the grounding pin consists of a second metal, which has a lower coefficient of thermal expansion than the first metal, in particular consists of niobium.

6. The feedthrough according to claim 5, wherein the grounding pin is embedded in the flange body by overmolding with the first metal.

7. The feedthrough according to claim 5, wherein the grounding pin is embedded in the flange body by heat-shrinking the flange body onto the grounding pin.

8. The feedthrough according to claim 7, wherein the grounding pin is embedded in the flange body by heat-shrinking an additively produced flange body onto the grounding pin.

9. A method for producing a feedthrough according to claim 1, wherein the grounding pin is fixed in a sequence of heat process steps comprising:
heating at least one material of the feedthrough flange or insulating element with simultaneous insertion, or insertion following the heating, of the grounding pin directly into the material of the feedthrough flange or insulating element or into an opening formed there previously, and the subsequent cooling of the material with grounding pin arranged therein.

10. The method according to claim 9, wherein the insulating element is formed as a solid ceramic insulating body and has a recess matched to the form of the grounding pin, the grounding pin is inserted into the insulating body in the green state of said insulating body, and the insulating body with inserted grounding pin is finished in a sintering process.

11. The method according to claim 9, wherein the grounding pin is inserted into an additively/generatively fabricated feedthrough flange prior to the debinding or sintering in a matched recess/opening, and the feedthrough flange with the inserted grounding pin is finished in a sintering process.

12. The method according to claim 9, wherein the grounding pin is placed in an is injection mold in order to form the feedthrough flange or insulating element and is overmolded by a plastic material introduced into the mold.

13. The method according to claim 9, wherein the grounding pin is placed into a metal injection mold in order to form the feedthrough flange and the mold is then filled with a liquefied material, and the grounding pin is overmolded by the metal.

14. The method according to claim 9, wherein a pre-fabricated feedthrough flange made of a first metal, in which a recess for receiving the grounding pin is formed, is heated and the grounding pin is introduced in the heated state into the recess, and the feedthrough flange with inserted grounding pin is then cooled in such a way that the grounding pin is shrunk into the feedthrough flange.

15. A medical electronic device comprising a feedthrough according to claim 1, in particular formed as a cardiac pacemaker, cardioverter, or cochlear implant.
